(19) United States
(12) Patent Application Publication
(10) Pub. No.: US 2008/0319163 A1
(43) Pub. Date: Dec. 25, 2008

(54) Method for Isolating and Purifying Immuno-Modulating Polypeptide from Cow Placenta

(76) Inventors: Yuliang Wang, Shijiazhuang (CN); Qinghai Sheng, Shijiazhuang (CN); Xinping Fang, Shijiazhuang (CN); Wenshui Xia, Shijiazhuang (CN); Hongmei Zhai, Shijiazhuang (CN); Peng Wei, Shijiazhuang (CN)

Correspondence Address: Michael L. Crapenhoft;c/o Hiroe and Associates, 4-3, Usa 3-chome, Gifu City, 500-8368, omitted

(21) Appl. No.: 11/911,201

(22) PCT Filed: Feb. 14, 2006
(86) PCT No.: PCT/CN2006/000219

§ 371 Date: May 12, 2008

(30) Foreign Application Priority Data
Apr. 21, 2005 (CN) 200510012463.0

(51) Int. Cl. C07K 7/06

(52) U.S. Cl. 530/328

(57) ABSTRACT

The present invention provides a method for isolating and purifying immuno-modulating polypeptide from cow placenta, which is characterized by using the steps of anion-exchange chromatography, gel exclusion chromatography and reverse-phase high performance liquid chromatography to isolate and purify immuno-modulating polypeptide from cow placenta, identifying its activity of stimulating lymphocyte proliferation in vitro by MTT method, then determining its molecular weight by MALDI-TOF-MS, its isoelectric point by CIEF and its amino acid sequence with analyzer for protein sequencing. Since the obtained immuno-modulating polypeptide by the method according to the present invention has more than 90% purity, its bioactivity can reach medicinal standards.

### TECHNICAL FIELD

The present invention relates to biology and medicine extraction field. More particularly, it relates to a method for isolating and purifying immuno-modulating polypeptide from cow placenta.

### BACKGROUND ART

With more people change their life to modern life style, the decline of the immunity would cause diseases. Enhancing the immunity is one of the essential approaches for resisting fatigue, improving work efficiency and preventing diseases.

The research of placenta immuno-modulating factor, the active components extracted from healthy puerperal placenta originated from China. In 1985, LIU Yuexin (Preparation and Study on Placenta Factor—A New Immunomodulator, Chinese Journal of Immunology,1985,1(5)) disclosed extracting a small molecular active substance from healthy puerpera placenta by homogenation-dialysis for the first time, and named the active substance as placenta factor. In 1994, HUANG Chuhua et al. (Laboratory Study of Placenta Polypeptide on Physical and Chemical Properties and Bioactivity, PHARMACEUTICAL BIOTECHNOLOGY, 1995,2(2)) obtained placenta injection, through the following steps: washing and cutting the fresh placenta, preparing homogenate with 2 times saline, centrifugal precipitating, ultrafiltering supernatants by ultrafiltration membrane with molecular weight cut-offs of 10,000, and sterilizing and dispensing the filtrate. In 1996, ZHANG Xuerong et al. (Experimental Study on Placenta Immuno-modulating Factor, GUANGXI SCIENCES,1996,3 ) disclosed placenta injection was obtained by cutting off anadesma of the placenta, washing with aseptic water, cutting and weighing, preparing homogenation by high-speed tissue homogenizer with 1:1 saline, then freezing the homogenation at −20° C. in refrigerator for more than 48 h, defrosting in water bath at 25° C., phacofragmentating for 15 min, dealing with poly frosted-defrosted and centrifugal precipitating at low temperature, dialyzing supernatants and filtering with membrane. In 2001, XU Daidi et al.(Preparing Placenta Immuno-modulating Factor, Guangxi Medical Journal,2001,8) prepared the placenta immuno-modulating factor referring to LIU Yuexin.

These researches show that, the main component of human placenta immuno-modulating factor is small molecular polypeptide. Large amount of animal experiments and clinic applications indicate that, placenta immuno-modulating factor is safety, nontoxic, no antigenicity, the activity would not decline for inducing antibody with long term use, and the placenta immuno-modulating factor is a safe high-efficient immunoregulator and immunostimulants. However, the present researches about placenta immuno-modulating factor mostly focus on its function and application, the reports on how to isolate and purify its main active components are rarely seen.

As a new kind of placenta resource, cow placenta, is similar with human in origin structure and components although differences between them are exit exactly. So it is important both on theory and technology that isolating and purifying immuno-modulating factor from cow placenta with different technology referring to the research on human placenta immuno-modulating factor.

### DISCLOSURE OF THE INVENTION

### Technical Problem

It is an object of the present invention to provide a method for extracting immuno-modulating polypeptide from cow placenta.

It is another object of the present invention to provide immuno-modulating polypeptide extracted from cow placenta.

### DESCRIPTION OF THE INVENTION

It is known that ion exchange chromatography is carried out in the system that employs ion exchange agent as stationary phase and liquid as mobile phase. Ion exchange agent comprises matrix, ion groups and counterion. The reaction between ion exchange agent and ion or ionic compounds in the liquid is mainly base on ion exchanging or base on absorption between ion groups of ion exchange agent and ion or ion compounds in solution. Ion exchange chromatography is carried out based on the difference of charge states (or polarities). Appropriate ion exchange agent should be chosen by dissociation properties or charge states of the objective. The conditions of absorption and elution should be controlled, main of which as ionic strength and the value of pH of eluant. Then the components in the mixture are eluted from chromatography column sequentially according to the affinity.

Gel exclusion chromatography is carried out by the movements of vertical downward and nondirectional diffusion of the molecules during them flowing through the chromatography column at a low flow rate. Macromolecules have big diameters, spread in spaces between particles instead in gel particle's small holes, and move vertically faster during elution. However, small molecules not only diffuse into spaces between gel particles but also entry gel particles inside. During the vertical movement, the small molecules diffuse from inside of the particle to spaces between the particles then entry the other particle inside. The movements of diffusion and entrance are continual. Therefore, the speed of vertical movement of small molecules is lower than that of macromolecules. As a result, macromolecules, medium molecules, small molecules are out of column sequentially, and separately successfully. The obvious advantage of gel exclusion chromatography is that the carrier it used is inert, and it is free of charge, weak absorbability, mild operate condition, wide operate temperature scope, no need of organic solvent, and has special advantage in maintaining physical and chemical properties of the compounds.

Reversed phase chromatography is widely used to isolating. In the system, solute is retained mainly by hydrophobic interaction. Hydrophobic interaction means that the interreaction between solute molecules or between solute molecules and water molecules is far less than that between water molecules where nonpolar solute is in the aqueous solution. Therefore, solute molecules can be removed from solution easily. Different compounds have different retention times for different hydrophobic properties, and the compounds are isolated as a result.

The present inventor have done lots of research, and combined these three methods creatively, which combined the anion exchange chromatography, gel exclusion chromatography and reversed phase high performance liquid chromatography together, to extract immuno-modulating polypeptide from cow placenta, determined molecular weight and isoelectric point and sequence of the isolated and purified immuno-modulating polypeptide.

The present invention relates to immuno-modulating polypeptide isolated from cow placenta. The characteristics of the immuno-modulating polypeptide are as follows: it has a molecular weight of 2133.52Da determined by MALDI-TOF-MS (matrix assisted laser desorption ionization time of flight mass spectrometry), an isoelectric point of 3.82 determined by CIEF ( Capillary isoelectric focusing), and a sequence of Tyr-X-Phe-Leu-Gly-Leu-Pro-Gly-X-Thr determined by 491-protein sequencer (Applied Biosystems, USA), wherein X is an amino acid.

The present invention also relates to a method for preparing immuno-modulating polypeptide isolated from cow placenta. The said method comprises the following steps known to those skilled in the art: washing and cutting fresh cow placenta, adding phosphate buffer of pH6.8-7.5 which 2 times (w/vol.) of placenta, preparing homogenate, centrifugating at 12000r/m, precipitating, ultrafiltering supernatants by ultrafiltration membrane with molecular weight cut-offs of 10,000, desalinating with aromatic polyamide nanofiltration membrane and freeze-dying, thus the lyophilized powder sample containing immuno-modulating polypeptide is obtained. The method also comprises dissolving the said lyophilized powder in phosphate buffer, using anion exchange chromatography, gel exclusion chromatography and reverse phase high performance liquid chromatography to isolate the solution, determining the active substance collected from reverse phase high performance liquid chromatography by RP-HPLC and capillary electrophoresis and the results both were single peak, then determining the molecular weight by MALDI-TOF-MS, isoelectric point by CIEF, and peptide sequence by protein sequencer.

According to an embodiment of the invention, the said anion exchange chromatography is performed under that dissolving 4-10:1(w:vol) the said lyophilized powder in phosphate buffer of 10-30 mmol/L pH6.8-7.5 as solution sample, loading the solution sample onto anion exchange column at the flow rate of 0.5-1.5 mL/min, gradient eluting at the flow rate of 0.5-1.5 mL/min, wherein eluting solution A is phosphate buffer of 10-30 mol/L, eluting solution B is the solution prepared by adding 0.5-1.5 mol/L NaCl solution to solution A, eluting at 0-600 min solution A and 600-1000 min solution B of 0-100%. Determining by MTT method through stimulating lymphocyte proliferation in vitro, screening, and the components containing immuno-modulating polypeptide is collected after separated by anion exchange chromatography.

Preferably, the ratio of the said lyophilized powder to the buffer used to dissolve the powder is 4-8:1 (w:vol.), more preferably, the ratio is 5-8:1.

Also, the buffer employed to dissolve the lyophilized powder according to the present invention is one or more solution chosen from Na₂HPO₄-NaH₂PO₄, K₂HPO₄-KH₂PO₄, K₂HPO₄-NaH₂PO₄ and/or Na₂HPO₄-KH₂PO₄ solution.

According to another embodiment of the present invention, the said lyophilized powder solution sample is 10-30 mmol/L pH 6.8-7.5 Na₂HPO₄-NaH₂PO₄ buffer.

Also, the eluting solution A employed by anion exchange chromatography is one or more solution chosen from Na₂HPO₄-NaH₂PO₄, K₂HPO₄-KH₂PO₄, K₂HPO₄-NaH₂PO₄ and Na₂HPO₄-KH₂PO₄ eluting solution.

According to another embodiment of the present invention, the said gel exclusion chromatography is performed under that loading the immuno-modulating polypeptide component collected from anion exchange chromatography onto the gel exclusion column directly at the flow rate of 8-12 mL/h, the mobile phase being phosphate buffer of 10-30 mmol/L pH6.8-7.5, eluting at flow rate of 8-12 mL/h with equal gradient elution, determining by MTT method through stimulating lymphocyte proliferation in vitro, screening, collecting the components containing immuno-modulating polypeptide after separated by anion exchange chromatography.

The said gel exclusion column has a length/diameter ratio of 60-90, and the volume of sample reaches to 1-10% of that of column bed.

Preferably, the said mobile phase used in gel exclusion column is one or more solutions chosen from Na₂HPO₄-NaH₂PO₄, K₂HPO₄-KH₂PO₄, K₂HPO₄-NaH₂PO₄ and Na₂HPO₄-KH₂PO₄ solution.

According to another embodiment of the present invention, the said reverse phase high performance liquid chromatography is performed under that loading the immuno-modulating polypeptide component collected from gel exclusion chromatography onto the reverse phase high performance liquid chromatography column directly at the flow rate of 0.8-1.2 mL/min, the mobile phase A being 5-10% Acetonitrile with 0.01-0.1% trifluoroacetic acid in, the mobile phase B being 40-60% Acetonitrile with 0.01-0.1% trifluoroacetic acid in, gradient eluting at flow rate of 0.8-1.2 mL/h with 0-8 min solution A, 8-12 min solution B(0-100%), 12-16 min solution B.

The said reverse phase high performance liquid chromatography column has a length/diameter ratio of 25-50, and the volume of sample reaches to 1-10% of that of column bed.

Preferably, the said mobile phase A used in the present invention is 5-8% Acetonitrile with 0.02-0.08% trifluoroacetic acid in, and mobile phase B is 40-60% Acetonitrile with 0.02-0.08% trifluoroacetic acid in.

According to another embodiment of the present invention, the said anion exchange column is equipped with anion exchange medias chosen from agarose DEAE Sepharose CL-6B, DEAE-Sepharose FF or glucan DEAE-Sephadex A-25 or DEAE-Sephadex A-50 functionalized with diethylaminoethyl.

Preferably, the said anion exchange column is equipped with DEAE Sepharose CL-6B, DEAE-Sepharose FF or DEAE-Sephadex A-25.

According to another embodiment of the present invention, the said gel exclusion chromatography column is glucan Sephadex G-25, Sephadex G-50 or Polyacrylamine gel Bio-gel-P-4, Bio-gel-P-6 or Bio-gel-P-10 with inactive porous net-like constructure, and separates substances in the protein mixture based on the sizes of molecules.

Preferably, the said gel exclusion chromatography column is a column with a media chosen from Sephadex G-25, Sephadex G-50, Bio-gel-P-4 and Bio-gel-P-6.

According to another embodiment of the present invention, the said reverse phase high performance liquid chromatography column is Sephasil peptide C 18 or Polymer C18 packing with ODS (ctadecylsilyl-silica).

### Advantageous Effect

The purity of immuno-modulating polypeptide from the present invention is higher than 90%, the bioactivity of which meets the standard of medicine preparing.

The immuno-modulating polypeptide from the present invention provides plenty of stuff for preparing healthy food with immuno-modulating activity.

The industry yield of immuno-modulating polypeptide from the present invention is 1%, 1 g immuno-modulating polypeptide from 100 g cow placenta (dry basis based).

### DESCRIPTION OF DRAWINGS

FIG. 1 is the chromatogram from the separation of immuno-modulating polypeptide in one embodiment of present invention on DEAE Sepharose CL-6B anion exchange column.

Peak 1a in FIG. 1 is the component containing immuno-modulating polypeptide.

FIG. 2 is the bar diagram of 4 components for stimulating lymphocyte proliferation in vitro, which separated from immuno-modulating polypeptide in one embodiment of present invention on DEAE Sepharose CL-6B anion exchange column.

Component 1a in FIG. 2 is the component containing immuno-modulating polypeptide.

FIG. 3 is the chromatogram from the separation of immuno-modulating polypeptide in one embodiment of present invention on Sephadex G-25 gel exclusion chromatography column.

Peak 1b in FIG. 3 is the component containing immuno-modulating polypeptide.

FIG. 4 is the bar diagram of 2 components for stimulating lymphocyte proliferation in vitro, which separated from immuno-modulating polypeptide in one embodiment of present invention on Sephadex G-25 gel exclusion chromatography column.

Component 1b in FIG. 4 is the component containing immuno-modulating polypeptide.

FIG. 5 is the chromatogram from the separation of immuno-modulating polypeptide in one embodiment of present invention on Sephasil peptide C₁₈ reversed phase high performance liquid chromatography column.

Peak 4 in FIG. 5 is the component containing immuno-modulating polypeptide.

FIG. 6 is the bar diagram of 7 components for stimulating lymphocyte proliferation in vitro, which separated from immuno-modulating polypeptide in one embodiment of present invention on Sephasil peptide C₁₈ reversed phase high performance liquid chromatography column.

Component 4 in FIG. 6 is the component containing immuno-modulating polypeptide.

FIG. 7 shows the purity of immuno-modulating polypeptide in one embodiment of present invention determined by Sephasil peptide C₁₈ reversed phase high performance liquid chromatography. The purity is higher than 90%.

FIG. 8 shows the purity of immuno-modulating polypeptide in one embodiment of present invention determined by capillary electrophoresis.

### DETAILED DESCRIPTION OF THE EMBODIMENTS

In order to understand the present invention more clearly, hereinafter, preferred embodiments of the present invention will be described in detail with reference to accompanying drawings.

### Detailed Description of the Experiment Method:

1) Stimulating Lymphocyte Proliferation in vitro

Culturing Cells
1 Preparing Spleen Lymphocyte Suspension:

Remove out the mice spleen under sterile condition, separate lymphocyte by lymphocyte separation medium, regulate the cell concentration to 1×10⁶/ml with complete 1640 culture solution.
2 Experiment Arrangement:

Set control group and experiment group separately to identify the activity of stimulating spleen lymphocyte proliferation by every component.
3. Experiment Method:

Add 100 μL mice spleen lymphocyte suspension (1×10⁶/mL) to 96 well cultured plates. Add 100 μL saline/sample to control group and experiment group separately. Mix to homogenous gently, culture in incubator for 68 h at 37° C. under 5% CO₂. Add 20 μl MTT to every well under sterile condition, Mix to homogenous gently, continue to culture for 4 h, add 100 μl dimethyl sulfoxide to every well, keep at room temperature for 10 min, then determine OD of every well by ELISA at 570 nm.
4. Data Processing

The spleen lymphocyte proliferation rate of experiment group is calculated by the following formula:

$$Spleen lymphocyte proliferation rate ( \% ) = \frac{{A}_{test} - {A}_{control}}{{A}_{test}} \times 100 \%$$

The value of P is calculated by variance analysis, and significant difference is calculate by T test.

2) Determining molecular weight of immuno-modulating polypeptide by MALDI-TOF-MS.

3) Determining isoelectric point of immuno-modulating polypeptide by CIEF.

4) Determining sequence of immuno-modulating polypeptide by protein sequencer.

5) Determining by RP-HPLC and capillary electrophoresis.

The foresaid methods 2)-5) are the normal methods known as those skilled in the art.

### EXAMPLES

### Example 1

### A. Placenta Preconditioning

Washed and cut fresh cow placenta, added phosphate buffer of pH7.0 which 2 times (w/v) of placenta, prepared homogenate, centrifugated at 12000 r/m for 3 min, precipitated, ultrafiltered supernatants by ultrafiltration membrane with molecular weight cut-offs of 10,000, desalinated with aromatic polyamide nanofiltration membrane and freeze-dried, thus the lyophilized powder sample containing immuno-modulating polypeptide was obtained.

### B. Anion Exchange Chromatography

Dissolved 30 mg lyophilized powder in 5 ml pH7.4 phosphate buffer with concentration of 20 mmol/L Na₂HPO₄-NaH₂PO₄, loaded the solution sample onto 2.6×35 cm DEAE Sepharose CL-6B anion exchange column at the flow rate of 1 ml/min, then gradient eluted, wherein eluting solution A was 20 mmol/L Na₂HPO₄-NaH₂PO₄ phosphate buffer, eluting solution B was 20 mmol/L Na₂HPO₄-NaH₂PO₄ phosphate buffer with 1 mol/L NaCl solution added in, and eluted at 0-600 min solution A and 600-1000 min solution B of 0-100%.( 0-100% meant from 100% solution A and 0% solution B to 0% solution A and 100% solution B).

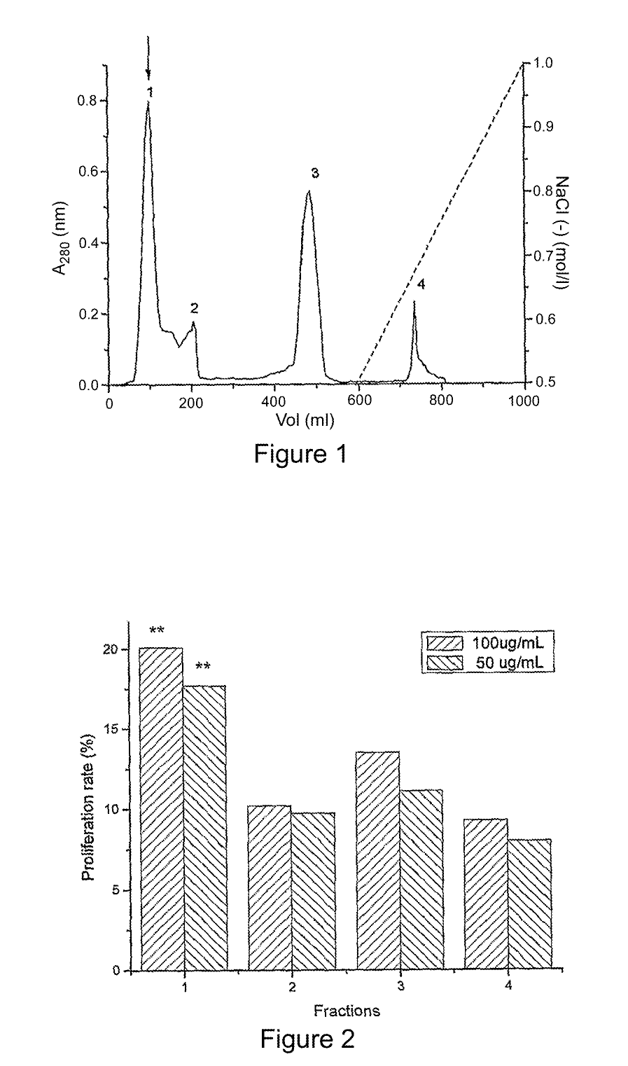

The anion exchange chromatogram showed in FIG. 1. The activity determined by stimulating lymphocyte proliferation in vitro showed in FIG. 2. The activity determining result showed that the composition from anion exchange chromatography contained immuno-modulating polypeptide (the peak that arrow indicated).

### C. Gel Exclusion Chromatography

Loaded foresaid immuno-modulating polypeptide component 1a collected from anion exchange chromatography onto 1.0×75 cm Sephadex G-25 gel exclusion column with the flow rate of 8 mL/h. The mobile phase was 20 mmol/L pH7.4 Na₂HPO₄-NaH₂PO₄ phosphate buffer. Equal gradient eluted.

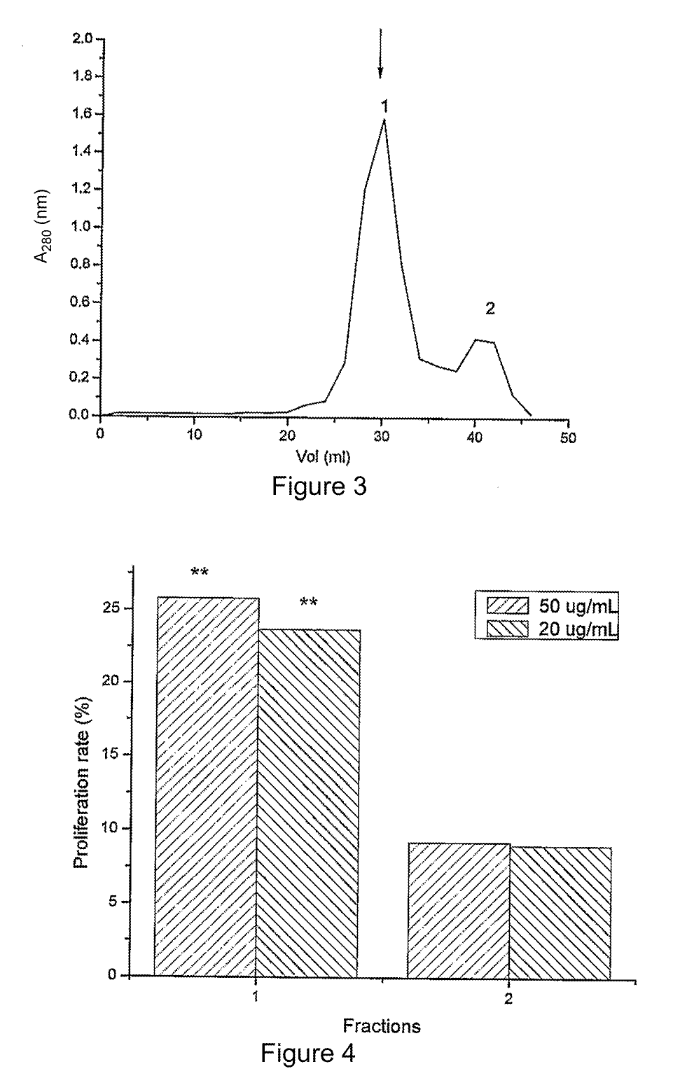

The gel exclusion chromatogram showed in FIG. 3. The activity determined by stimulating lymphocyte proliferation in vitro showed in FIG. 4. The activity determining result showed that the composition from gel exclusion chromatography contained immuno-modulating polypeptide (the peak that arrow indicated).

### D. Reverse Phase High Performance Liquid Chromatography

Loaded foresaid immuno-modulating polypeptide component 1b collected from gel exclusion chromatography onto Sephasil peptide C₁₈ reverse phase high performance liquid chromatography column with the flow rate of 1 mL/min. The mobile phase A was 10% Acetonitrile with 0.05% trifluoroacetic acid in, the mobile phase B was 60% Acetonitrile with 0.05% trifluoroacetic acid in, gradient eluted with 0-8 min solution A, 8-12 min solution B(0-100%), then 12-16 min solution B.

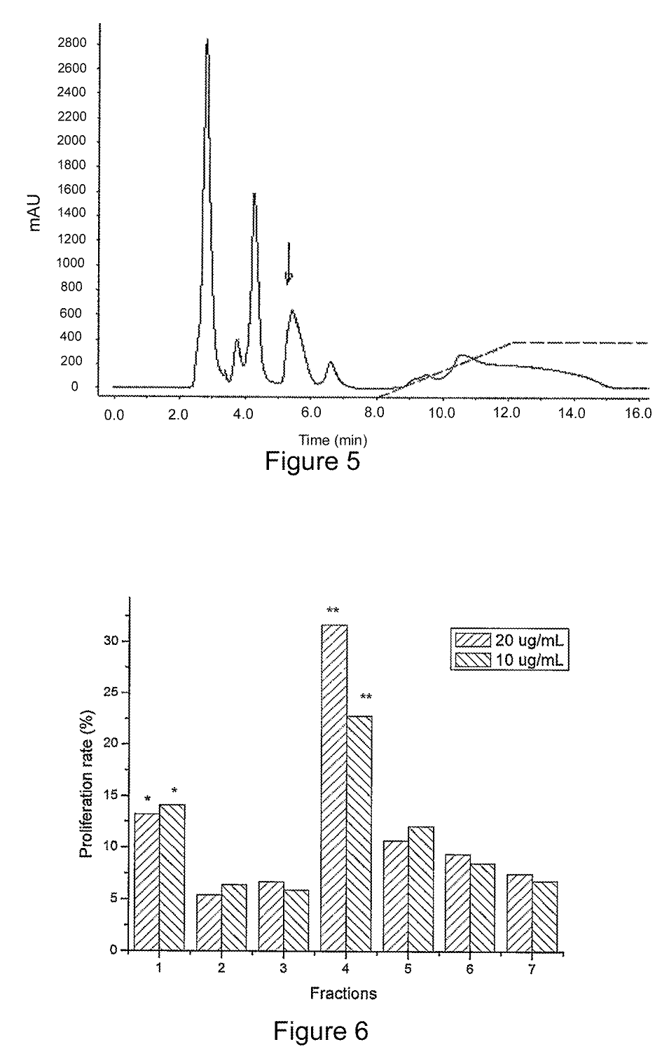

The reverse phase high performance liquid chromatogram showed in FIG. 5. The activity determined by stimulating lymphocyte proliferation in vitro showed in FIG. 6. The activity determining result showed that the component 4 was high-purity immuno-modulating polypeptide.

### E. Identifying and Analyzing

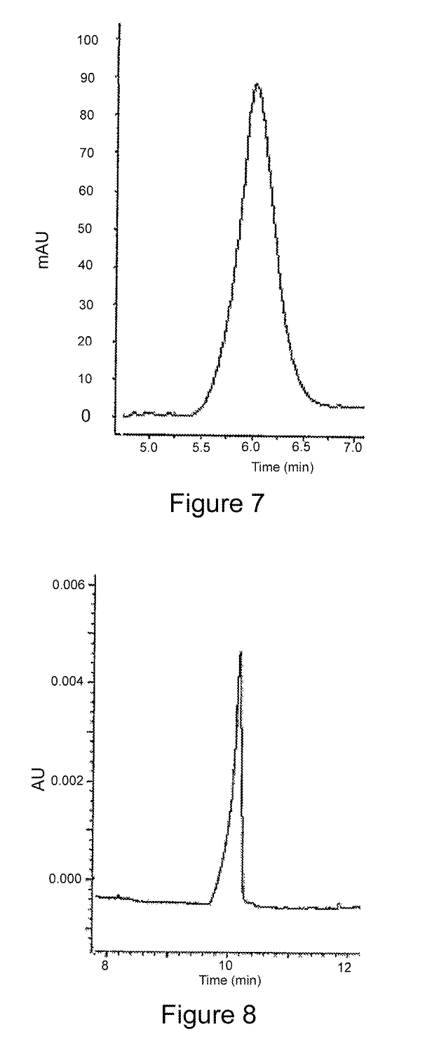

Desalinated component 4 with nanofiltration membrane and freeze-dried to obtain immuno-modulating polypeptide product. Identified the product purity by Sephasil peptide C₁₈ RP-HPLC and capillary electrophoresis. The results showed in FIG. 7 and FIG. 8 separately, and both were single peak.

The molecular weight of immuno-modulating polypeptide determined by MALDI-TOF-MS was 2133.52 Da.

The peptide sequence of immuno-modulating polypeptide determined by 491-protein sequencer was Tyr-X-Phe-Leu-Gly-Leu-Pro-Gly-X-Thr (X, an amino acid).

### Example 2

### A. Placenta Preconditioning

Washed and cut fresh cow placenta, added phosphate buffer of pH7.0 which 2 times (w/v) of placenta, prepared homogenate, centrifugated at 12000 r/m, precipitated, ultrafiltered supernatants by ultrafiltration membrane with molecular weight cut-offs of 10,000, desalinated with aromatic polyamide nanofiltration membrane and freeze-dried, thus the lyophilized powder sample containing immuno-modulating polypeptide was obtained.

### B. Anion Exchange Chromatography

Dissolved 30 mg lyophilized powder in 5 ml pH6.8 phosphate buffer with concentration of 20 mmol/L Na₂HPO₄-NaH₂PO₄, loaded the solution sample on 2.6×35 cm DEAE Sepharose CL-6B anion exchange column at the flow rate of 1 ml/min, then gradient eluted, wherein eluting solution A was 20 mmol/L Na₂HPO₄-NaH₂PO₄ phosphate buffer, eluting solution B was 20 mmol/L Na₂HPO₄-NaH₂PO₄ phosphate buffer with 1 mol/L NaCl solution added in, and eluted at 0-600 min solution A and 600-1000 min solution B of 0-100%.

The anion exchange chromatogram and the activity determined by stimulating lymphocyte proliferation in vitro were similar with FIG. 1 and FIG. 2 separately. The activity determining result showed that the composition from anion exchange chromatography contained immuno-modulating polypeptide 1(the peak that arrow indicated).

### C. Gel Exclusion Chromatography

Loaded foresaid immuno-modulating polypeptide component 1a collected from anion exchange chromatography onto 1.0×75 cm Sephadex G-25 gel exclusion column with the flow rate of 10 mL/h. The mobile phase was 20 mmol/L pH6.8 Na₂HPO₄-NaH₂PO₄ phosphate buffer. Equal gradient eluted.

The gel exclusion chromatogram and the activity determined by stimulating lymphocyte proliferation in vitro were similar with FIG.3 and FIG. 4 separately. The activity determining result showed that the composition from gel exclusion chromatography contained immuno-modulating polypeptide (the peak that arrow indicated).

### D. Reverse Phase High Performance Liquid Chromatography

Loaded foresaid immuno-modulating polypeptide component 1b collected from gel exclusion chromatography onto Sephasil peptide C₁₈ reverse phase high performance liquid chromatography column with the flow rate of 1 mL/min. The mobile phase A was 8% Acetonitrile with 0.05% trifluoroacetic acid in, the mobile phase B was 55% Acetonitrile with 0.05% trifluoroacetic acid in, gradient eluted with 0-8 min solution A, 8-12 min solution B(0-100%), then 12-16 min solution B.

The reverse phase high performance liquid chromatogram and the activity determined by stimulating lymphocyte proliferation in vitro were similar with FIG. 5 and FIG. 6 separately. The activity determining result showed that the component 4 was high-purity immuno-modulating polypeptide.

### E. Identifying and Analyzing

Desalinated component 4 with nanofiltration membrane and freeze-dried to obtain immuno-modulating polypeptide product. Identified the product purity by Sephasil peptide C₁₈ RP-HPLC and capillary electrophoresis. The results were similar with FIG. 7 and FIG. 8 separately, and both were single peak.

The molecular weight of immuno-modulating polypeptide determined by MALDI-TOF-MS was 2133.52 Da.

The isoelectric point of immuno-modulating polypeptide determined by CIEF was 3.82.

The peptide sequence of immuno-modulating polypeptide determined by 491-protein sequencer was Tyr-X-Phe-Leu-Gly-Leu-Pro-Gly-X-Thr (X, an amino acid).

### Example 3

### A. Placenta Preconditioning

Washed and cut fresh cow placenta, added phosphate buffer of pH7.0 which 2 times (w/v) of placenta, prepared homogenate, centrifugated at 12000 r/m, precipitated, ultrafiltered supernatants by ultrafiltration membrane with molecular weight cut-offs of 10,000, desalinated with aromatic polyamide nanofiltration membrane and freeze-dried, thus the lyophilized powder sample containing immuno-modulating polypeptide was obtained.

### B. Anion Exchange Chromatography

Dissolved 30 mg lyophilized powder in 5 ml pH7.2 phosphate buffer with concentration of 20 mmol/L Na₂HPO₄-NaH₂PO₄, loaded the solution sample onto 2.6×35 cm DEAE Sepharose CL-6B anion exchange column at the flow rate of 1 ml/min, then gradient eluted, wherein eluting solution A was 20 mmol/L Na₂HPO₄-NaH₂PO₄ phosphate buffer, eluting solution B was 20 mmol/L Na₂HPO₄-NaH₂PO₄ phosphate buffer with 1 mol/L NaCl solution added in, and eluted at 0-600 min solution A and 600-1000 min solution B of 0-100%.

The anion exchange chromatogram and the activity determined by stimulating lymphocyte proliferation in vitro were similar with FIG. 1 and FIG. 2 separately. The activity determining result showed that the composition from anion exchange chromatography contained immuno-modulating polypeptide 1(the peak that arrow indicated).

### C. Gel Exclusion Chromatography

Loaded foresaid immuno-modulating polypeptide component 1a collected from anion exchange chromatography onto 1.0×75 cm Sephadex G-25 gel exclusion column with the flow rate of 12 mL/h. The mobile phase was 20 mmol/L pH7.2 Na₂HPO₄-NaH₂PO₄ phosphate buffer. Equal gradient eluted.

The gel exclusion chromatogram and the activity determined by stimulating lymphocyte proliferation in vitro were similar with FIG. 3 and FIG. 4 separately. The activity determining result showed that the composition from gel exclusion chromatography contained immuno-modulating polypeptide (the peak that arrow indicated).

### D. Reverse Phase High Performance Liquid Chromatography

Loaded foresaid immuno-modulating polypeptide component 1b collected from gel exclusion chromatography onto Sephasil peptide C₁₈ reverse phase high performance liquid chromatography column with the flow rate of 1 mL/min. The mobile phase A was 5% Acetonitrile with 0.05% trifluoroacetic acid in, the mobile phase B was 45% Acetonitrile with 0.05% trifluoroacetic acid in, gradient eluted with 0-8 min solution A, 8-12 min solution B(0-100%), then 12-16 min solution B.

The reverse phase high performance liquid chromatogram and the activity determined by stimulating lymphocyte proliferation in vitro were similar with FIG. 5 and FIG. 6 separately. The activity determining result showed that the component 4 was high-purity inmmuno-modulating polypeptide.

### E. Identifying and Analyzing

Desalinated component 4 with nanofiltration membrane and freeze-dried to obtain immuno-modulating polypeptide product. Identified the product purity by Sephasil peptide C₁₈ RP-HPLC and capillary electrophoresis. The results were similar with FIG. 7 and FIG. 8 separately, and both were single peak.

The molecular weight of immuno-modulating polypeptide determined by MALDI-TOF-MS was 2133.52 Da.

The isoelectric point of immuno-modulating polypeptide determined by CIEF was 3.82.

The peptide sequence of immuno-modulating polypeptide determined by 491-protein sequencer was Tyr-X-Phe-Leu-Gly-Leu-Pro-Gly-X-Thr (X, an amino acid).

### Example 4

### A. Placenta Preconditioning

Washed and cut fresh cow placenta, added phosphate buffer of pH7.0 which 2 times (w/v) of placenta, prepared homogenate, centrifugated at 12000 r/m, precipitated, ultrafiltered supernatants by ultrafiltration membrane with molecular weight cut-offs of 10,000, desalinated with aromatic polyamide nanofiltration membrane and freeze-dried, thus the lyophilized powder sample containing immuno-modulating polypeptide was obtained.

### B. Anion Exchange Chromatography

Dissolved 30 mg lyophilized powder in 5 ml pH7.2 phosphate buffer with concentration of 20 mmol/L Na₂HPO₄-NaH₂PO₄, loaded the solution sample on 2.6×35 cm DEAE Sepharose CL-6B anion exchange column at the flow rate of 1 ml/min, then gradient eluted, wherein eluting solution A was 20 mmol/L Na₂HPO₄-NaH₂PO₄ phosphate buffer, eluting solution B was 20 mmol/L Na₂HPO₄-NaH₂PO₄ phosphate buffer with 1 mol/L NaCl solution added in, and eluted at 0-600min solution A and 600-1000 min solution B of 0-100%.

The anion exchange chromatogram and the activity determined by stimulating lymphocyte proliferation in vitro were similar with FIG. 1 and FIG. 2 separately. The activity determining result showed that the composition from anion exchange chromatography contained immuno-modulating polypeptide 1(the peak that arrow indicated).

### C. Gel Exclusion Chromatography

Loaded foresaid immuno-modulating polypeptide component 1a collected from anion exchange chromatography onto 1.0×75 cm Sephadex G-25 gel exclusion column with the flow rate of 12 mL/h. The mobile phase was 20 mmol/L pH7.2 Na₂HPO₄-NaH₂PO₄ phosphate buffer. Equal gradient eluted.

The gel exclusion chromatogram and the activity determined by stimulating lymphocyte proliferation in vitro were similar with FIG. 3 and FIG. 4 separately. The activity determining result showed that the composition from gel exclusion chromatography contained immuno-modulating polypeptide (the peak that arrow indicated).

### D. Reverse Phase High Performance Liquid Chromatography

Loaded foresaid immuno-modulating polypeptide component 1b collected from gel exclusion chromatography onto Sephasil peptide C₁₈ reverse phase high performance liquid chromatography column with the flow rate of 1 mL/min. The mobile phase A was 5% Acetonitrile with 0.05% trifluoroacetic acid in, the mobile phase B was 60% Acetonitrile with 0.05% trifluoroacetic acid in, gradient eluted with 0-8 min solution A, 8-12 min solution B(0-100%), then 12-16 min solution B.

The reverse phase high performance liquid chromatogram and the activity determined by stimulating lymphocyte proliferation in vitro were similar with FIG. 5 and FIG. 6 separately. The activity determining result showed that the component 4 was high-purity immuno-modulating polypeptide.

### E. Identifying and Analyzing

Desalinated component 4 with nanofiltration membrane and freeze-dried to obtain immuno-modulating polypeptide product. Identified the product purity by Sephasil peptide C₁₈ RP-HPLC and capillary electrophoresis. The results were similar with FIG. 7 and FIG. 8 separately, and both were single peak.

The molecular weight of immuno-modulating polypeptide determined by MALDI-TOF-MS was 2133.52 Da.

The isoelectric point of immuno-modulating polypeptide determined by CIEF was 3.82.

The peptide sequence of immuno-modulating polypeptide determined by 491-protein sequencer was Tyr-X-Phe-Leu-Gly-Leu-Pro-Gly-X-Thr (X-an amino acid).

### Example 5

### A. Placenta Preconditioning

Washed and cut fresh cow placenta, added phosphate buffer of pH7.0 which 2 times (w/v) of placenta, prepared homogenate, centrifugated at 12000 r/m, precipitated, ultrafiltered supernatants by ultrafiltration membrane with molecular weight cut-offs of 10,000, desalinated with aromatic polyamide nanofiltration membrane and freeze-dried, thus the lyophilized powder sample containing immuno-modulating polypeptide was obtained.

### B. Anion Exchange Chromatography

Dissolved 30 mg lyophilized powder in 5 ml pH7.4 phosphate buffer with concentration of 20 mmol/L Na₂HPO₄-NaH₂PO₄, loaded the solution sample onto 2.6×35 cm DEAE Sepharose CL-6B anion exchange column at the flow rate of 1 ml/min, then gradient eluted, wherein eluting solution A was 20 mmol/L Na₂HPO₄-NaH₂PO₄ phosphate buffer, eluting solution B was 20 mmol/L Na₂HPO₄-NaH₂PO₄ phosphate buffer with 1 mol/L NaCl solution added in, and eluted at 0-600 min solution A and 600-1000 min solution B of 0-100%.( 0-100% meant from 100% solution A and 0% solution B to 0% solution A and 100% solution B).

The anion exchange chromatogram and the activity determined by stimulating lymphocyte proliferation in vitro were similar with FIG. 1 and FIG. 2 separately. The activity determining result showed that the composition from anion exchange chromatography contained immuno-modulating polypeptide 1(the peak that arrow indicated).

### C. Gel Exclusion Chromatography

Loaded foresaid immuno-modulating polypeptide component 1a collected from anion exchange chromatography onto 1.0×75 cm Sephadex G-25 gel exclusion column with the flow rate of 10 mL/h. The mobile phase was 20 mmol/L pH7.4 Na₂HPO₄-NaH₂PO₄ phosphate buffer. Equal gradient eluted.

The gel exclusion chromatogram and the activity determined by stimulating lymphocyte proliferation in vitro were similar with FIG. 3 and FIG. 4 separately. The activity determining result showed that the composition from gel exclusion chromatography contained immuno-modulating polypeptide (the peak that arrow indicated).

### D. Reverse Phase High Performance Liquid Chromatography

Loaded foresaid immuno-modulating polypeptide component 1b collected from gel exclusion chromatography onto Sephasil peptide C₁₈ reverse phase high performance liquid chromatography column with the flow rate of 1 mL/min. The mobile phase A was 10% Acetonitrile with 0.05% trifluoroacetic acid in, the mobile phase B was 40% Acetonitrile with 0.05% trifluoroacetic acid in, gradient eluted with 0-8 min solution A, 8-12 min solution B(0-100%), then 12-16 min solution B.

The reverse phase high performance liquid chromatogram was similar with FIG. 5, and the activity determined by stimulating lymphocyte proliferation in vitro was similar with FIG. 6. The activity determining result showed that the component 4 was high-purity immuno-modulating polypeptide.

### E. Identifying and Analyzing

Desalinated component 4 with nanofiltration membrane and freeze-dried to obtain immuno-modulating polypeptide product. Identified the product purity by Sephasil peptide C₁₈ RP-HPLC and capillary electrophoresis. The results were similar with FIG. 7 and FIG. 8 separately, and both were single peak.

The molecular weight of immuno-modulating polypeptide determined by MALDI-TOF-MS was 2133.52 Da.

The isoelectric point of immuno-modulating polypeptide determined by CIEF was 3.82.

The peptide sequence of immuno-modulating polypeptide determined by 491-protein sequencer was Tyr-X-Phe-Leu-Gly-Leu-Pro-Gly-X-Thr (X, an amino acid).

The solution B used by reverse phase high performance liquid chromatography was 40% Acetonitrile with 0.05% trifluoroacetic acid in; other operate conditions were as the same as example 1.

The foresaid embodiments are used to explain the method for separating immuno-modulating polypeptide from cow placenta, do not limit the scope of the present invention. However, it will be apparent to those skilled in the art that various modifications and variations can be made for carrying out the same purposes of the present invention, therein without departing from the spirit and scope of the invention.

What is claimed is:

1. An immuno-modulating polypeptide isolated from cow placenta, characterized in that the molecular weight of the immuno-modulating polypeptide is 2133.52 Da, isoelectric point is 3.82, and peptide sequence is Tyr-X-Phe-Leu-Gly-Leu-Pro-Gly-X-Thr, wherein X is an amino acid.

2. A method for preparing immuno-modulating polypeptide isolated from cow placenta comprising: removing out fresh cow placenta, watering, cutting, homogenating, centrifugating, ultrafiltering, desalinating with nanofiltration membrane and freeze-dried, characterized in that the method also comprises: dissolving lyophilized powder obtained by the said freeze-drying step in phosphate buffer, using anion exchange chromatography, gel exclusion chromatography and reverse phase high performance liquid chromatography to isolate the solution, desalinating the component collected from reverse phase high performance liquid chromatography with nanofiltration membrane and freeze-drying, determining the immuno-modulating polypeptide product by RP-HPLC and capillary electrophoresis and the results both were single peak, then determining the molecular weight by MALDI-TOF-MS, isoelectric point by CIEF, and peptide sequence by protein sequencer.

3. The method according to claim 2, characterized in that the said anion exchange chromatography is performed under that dissolving 4-10:1 (w:vol) the said lyophilized powder in phosphate buffer of 10-30 mmol/L pH6.8-7.5 as solution sample, loading the solution sample onto anion exchange column at the flow rate of 0.5-1.5 mL/min, gradient eluting at the flow rate of 0.5-1.5 mL/min, wherein eluting solution A is phosphate buffer of 10-30 mol/L, eluting solution B is the solution prepared by adding 0.5-1.5 mol/L NaCl solution to solution A, eluting at 0-600 min solution A and 600-1000 min solution B of 0-100%. Determining by MTT method through stimulating lymphocyte proliferation in vitro, screening, and the components containing immuno-modulating polypeptide is collected after separated by anion exchange chromatography.

4. The method according to claim 2, characterized in that the said gel exclusion chromatography is performed under that loading the immuno-modulating polypeptide component collected from anion exchange chromatography onto the gel exclusion column directly at the flow rate of 8-12 mL/h, the mobile phase being phosphate buffer of 10-30 mmol/L pH6.8-7.5, eluting at flow rate of 8-12 mL/h with equal gradient elution, determining by MTT method through stimulating lymphocyte proliferation in vitro, screening, collecting the components containing immuno-modulating polypeptide after separated by anion exchange chromatography.

5. The method according to claim 2, characterized in that the said reverse phase high performance liquid chromatography is performed under that loading the immuno-modulating polypeptide component collected from gel exclusion chromatography onto the reverse phase high performance liquid chromatography column directly at the flow rate of 0.8-1.2 mL/min, the mobile phase A being 5-10% Acetonitrile with 0.01-0.1% trifluoroacetic acid in, the mobile phase B being 40-60% Acetonitrile with 0.01-0.1% trifluoroacetic acid in, gradient eluting at flow rate of 0.8-1.2 mL/h with 0-8 min solution A, 8-12 min solution B(0-100%), 12-16 min solution B.

6. The method according to claim 3, characterized in that the said anion exchange column is equipped with anion exchange medias chosen from agarose DEAE Sepharose CL-6B,or DEAE-Sepharose FF or glucan DEAE-Sephadex A-25 or DEAE-Sephadex A-50 functionalized with diethylaminoethyl.

7. The method according to claim 4, characterized in that the said gel exclusion chromatography column is glucan Sephadex G-25, Sephadex G-50 or Polyacrylamine gel Bio-gel-P-4, Bio-gel-P-6 or Bio-gel-P-10 with inactive porous net-like constructure, and separates substances in the protein mixture based on the sizes of molecules.

8. The method according to claim 5, characterized in that the said reverse phase high performance liquid chromatography column is Sephasil peptide C₁₈ or Polymer C₁₈ packing with ODS reverse phase packing.

9. The method according to claim 3, characterized in that the said solution sample is 10-30 mmol/L pH 6.8-7.5Na₂HPO₄-NaH₂PO₄ buffer.
